(19) United States
(12) Patent Application Publication
(10) Pub. No.: US 2023/0058048 A1
(43) Pub. Date: Feb. 23, 2023

(54) SYSTEMS AND METHODS FOR INCREASING PRIOR AUTHORIZATION SUCCESS RATE FOR SPECIALTY DRUGS

(71) Applicant: Change Healthcare Holdings, LLC, Nashville, TN (US)

(72) Inventor: James Delbert Smith, Willow Park, TX (US)

(21) Appl. No.: 17/409,192

(22) Filed: Aug. 23, 2021

(51) Int. Cl. G16H 20/10; G16H 15/00; G06Q 10/10

(52) U.S. Cl. CPC G16H 20/10; G16H 15/00; G06Q 10/10

(57) ABSTRACT

A system for increasing the rate of claim approval for specialty drugs is provided. The system monitors the approval and disapproval rates for claims related to specialty medications for multiple providers (e.g., pharmacies) due to missing or incomplete prior authorization requests. When the approval rate for a particular drug for a provider falls below a threshold value due to incomplete or missing prior authorization requests, the system provides the manufacturer of the drug with training on how to complete prior authorization requests for the drug. For example, the provider may be invited to an online course, or instructors may offer to visit the provider to provide additional training. The drug manufacturers may reimburse the claim processing system for identifying those providers who could benefit from the training.

### BACKGROUND

Specialty drugs refers to a class of drugs that meets some or all of the following characteristics: prescribed for a complex or chronic medical condition; treats rare or orphan diseases; requires additional patient education, adherence, or support; has a high monthly cost, has unique store requirements; and is not stocked at many retail pharmacies. Because of there high costs and unusual requirements, many payor (e.g., insurance providers) require extensive paperwork and other documentation to receive prior authorizations for specialty drugs.

As may be appreciated, because of the difficulty is receiving prior authorization, many providers (e.g., pharmacies) either refuse to fill prescriptions for specialty drugs, or frequently fail in their attempts to receive prior authorization for specialty drugs. Accordingly, patients who may benefit from such drugs may not receive the drugs, may be forced to delay or skip doses of the drugs, or may switch to less effective drugs with less onerous prior authorization requirements.

### SUMMARY

In one embodiment, systems, and methods for increasing the rate of claim approval for specialty drugs are provided. A system monitors the approval and disapproval rates for claims related to specialty medications for multiple providers (e.g., pharmacies) due to missing or incomplete prior authorization requests. When the approval rate for a particular drug for a provider falls below a threshold value due to incomplete or missing prior approval requests, the system provides the manufacturer of the drug with training on how to complete prior authorization requests for the drug. For example, the provider may be invited to an online course, or instructors may offer to visit the provider to provide additional training. The drug manufacturers may reimburse the claim processing system for identifying those providers who could benefit from the training.

In an embodiment, a method is provided. The method includes receiving a claim from a provider by a computing device, wherein the claim is for a drug produced by a manufacturer; transmitting the claim to a claim processing system; receiving a claim denial response from the claim processing system; updating one or more metrics for the provider and the drug based on the claim denial response by the computing device; determining whether the one or more metrics fall below a threshold for the manufacturer and the drug by the computing device; and if it is determined that the one or more metrics fall below the threshold, initiating training for the provider by the computing device.

Embodiments may include some or all of the following features. The drug may be a specialty drug. The method may further include receiving compensation for the training by the manufacturer. At least one metric of the one or more metrics may be an approval rate. Initiating training may include providing a link to training materials to the provider. The provider may be a pharmacy or a physician. The training may be training on completing paperwork related to a prior authorization.

In an embodiment, a method may be provided. The method may include: monitoring acceptances and denials of claims due to prior authorization requests for a plurality of drugs associated with a manufacturer by a computing device, wherein each claim is associated with a provider of a plurality of providers; for each provider of the plurality of providers and each drug of the plurality of drugs, computing an acceptance rate for the claims for the drug and the provider by the computing device; determining that a computed acceptance rate for a first drug of the plurality drugs and a first provider of the plurality of providers falls below a threshold by the computing device; and in response to the determination, initiating training for the first provider on preparing prior authorization requests for the first drug by the computing device.

Embodiment may include some or all of the following features. The drug may be a specialty drug. The method may further include receiving compensation for the training by the manufacturer. Initiating training may include providing a link to training materials to the first provider. The method may further include receiving the training materials from the manufacturer. The first provider may be a pharmacy or a physician. The training may be training on completing paperwork related to the prior authorization request for the first drug. The method may further include providing a graphical user interface to the manufacturer; and displaying some or all of the computed acceptance rates for the plurality of drugs for some or all of the plurality of providers in the graphical user interface. The method may further include receiving the threshold from the manufacturer.

In an embodiment, a system is provided. The system includes at least one computing device and a memory. The memory stores instructions that when executed by the at least one computing device cause the at least one computing device to: receive a claim from a provider, wherein the claim is for a drug produced by a manufacturer; transmit the claim to a claim processing system; receive a claim denial response from the claim processing system; update one or more metrics for the provider and the drug based on the claim denial response; determine whether the one or more metrics fall below a threshold for the manufacturer and the drug; and if it is determined that the one or more metrics fall below the threshold, initiate training for the provider.

Embodiment may include some or all of the following features. The drug may be a specialty drug. The system may further receive compensation for the training by the manufacturer. The metric may be an approval rate.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying figures, which are incorporated herein and form part of the specification, illustrate a document attachment system and method. Together with the description, the figures further serve to explain the principles of the document attachment system and method described herein and thereby enable a person skilled in the pertinent art to make and use the document attachment system and method.

FIG. 1 is an example environment for improving approval rates for claims;

FIG. 2 is an illustration of an example graphical user interface for viewing information associated with claims;

FIG. 3 is an illustration of a method for providing training to providers to improve claim acceptance rates for specialty drugs;

FIG. 4 is an illustration of a method for compensating an entity for monitoring claim approval rates; and

FIG. 5 shows an exemplary computing environment in which example embodiments and aspects may be implemented.

### DETAILED DESCRIPTION

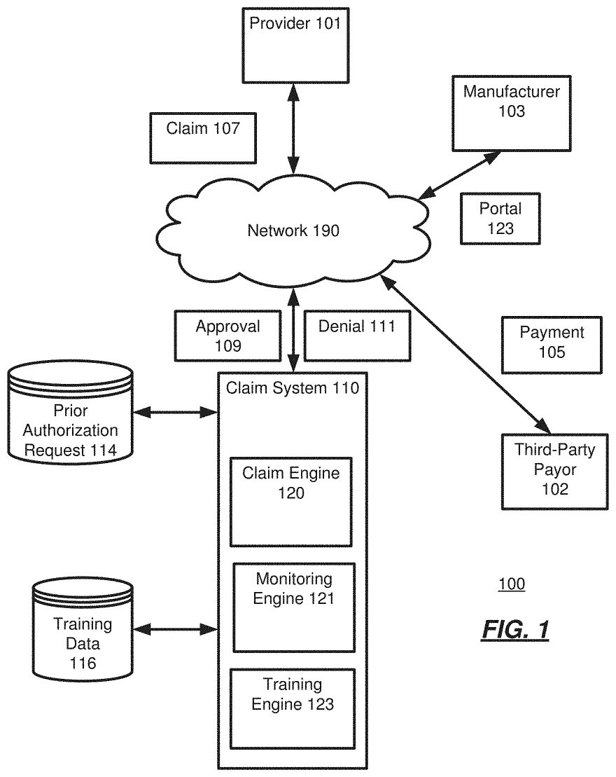

FIG. 1 is an example environment 100 for monitoring the approval rates of claims for specialty drugs. As shown, the environment 100 includes a provider 101, a claim system 110, a manufacturer 103, and a third-party-payor 102 communicating through a network 190. While only one provider 101, manufacturer 103, claim system 110 and third-party payor 102 is shown; it is contemplated that there may be multiple providers 101, third-party payors 102, and manufacturers 103 in the environment 100.

The provider 101 may submit a claim 107 for a specialty drug or medication. Examples of providers 101 may include pharmacies, physicians, or other medical providers. As described above, specialty drugs are a class of drugs that require prior authorization from third-party payors 102 in order to receive reimbursement for a claim 107. Often a prior authorization request requires extensive paperwork, making completing them time consuming and difficult for providers 101. As result, prior authorization requests are often not completed, or may be incorrectly completed, which may result in an associated patient not receiving the specialty drug.

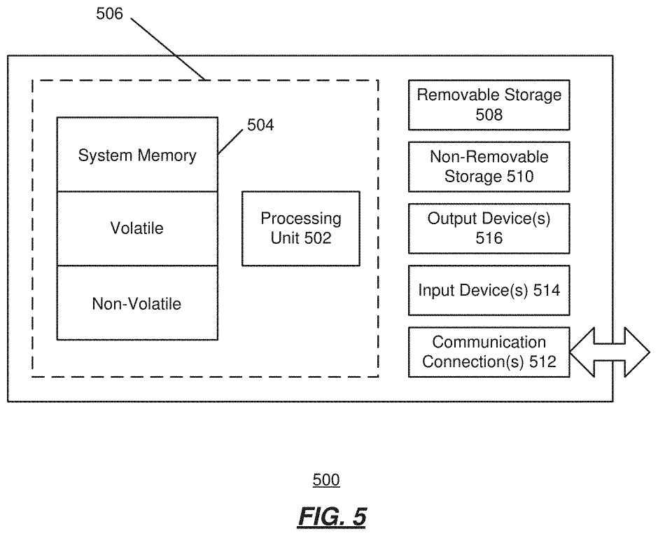

As shown the claim system 110 may include various components including a claim engine 120, a monitoring engine 121, and a training engine 123. More or fewer components may be supported. Depending on the embodiment, each of the engines 120, 121, and 123 may be implemented together or separately using one or more general purpose computing devices such as the computing device 500 illustrated with respect to FIG. 5. In addition, either engine may be implemented together or separately using a cloud-based computing environment.

The claim engine 120 may receive a claim 107 for a specialty drug, and as part of processing the claim 107 may determine if a prior authorization request 114 was received for the patient associated with the claim 107. A prior authorization request 114 for a specialty drug may include one or more forms that must be submitted by a doctor or other medical professional in order for a specialty drug to be paid for by a third party payor 102. The third-party payor 102 may be an insurance provider or a government entity, for example. Note that while the claim 107 is shown as being received electronically via a network, in some embodiments, some or all of the claims 107 may be received via other means such as fax, mail, or telephone.

When a claim 107 is received by the claim engine 120 for a specialty drug, the claim engine 120 may determine whether a prior authorization request 114 was received for the claim 107. If so, the claim engine 120 may approve the claim 107 and may provide an approval 109 to the provider 101. The provider 101 may then dispense the specialty drug to the patient or individual associated with the claim 107.

If the claim engine 120 determines that no prior authorization request 114 was received, or that a deficient or incomplete prior authorization request 114 was received, the claim engine 120 may deny the claim 107, and may provide a denial 111 to the provider 101. The denial 111 may indicate the reason for the denial 111 was due to the lack of a prior authorization request 114 or an incomplete prior authorization request 114.

The monitoring engine 121 may further track the acceptance or denial of claims 107 for specially drugs for each provider 101 and may generate various metrics about each provider 101. The metrics for a provider 101 may include, for each specialty drug, the number of claims 107 that were rejected due to missing or incomplete prior authorization requests 114.

The monitoring engine 121 may further provide a portal 123 through which a manufacturer 103 can view the metrics associated with each provider 101 for the specialty drugs associated with the manufacturer 103. In this way, the manufacturer 103 of a particular specialty drug may learn which providers 101 are having problems receiving prior authorization for their drugs.

In some embodiments, the portal 123 may be a website or application that the drug manufacturers 103 may pay a monthly fee or subscription to access through the network 190. Alternatively, the metrics in the portal 123 may be provided to the manufacturers 103 in a daily, weekly, or monthly email or report that is generated by the monitoring engine 121.

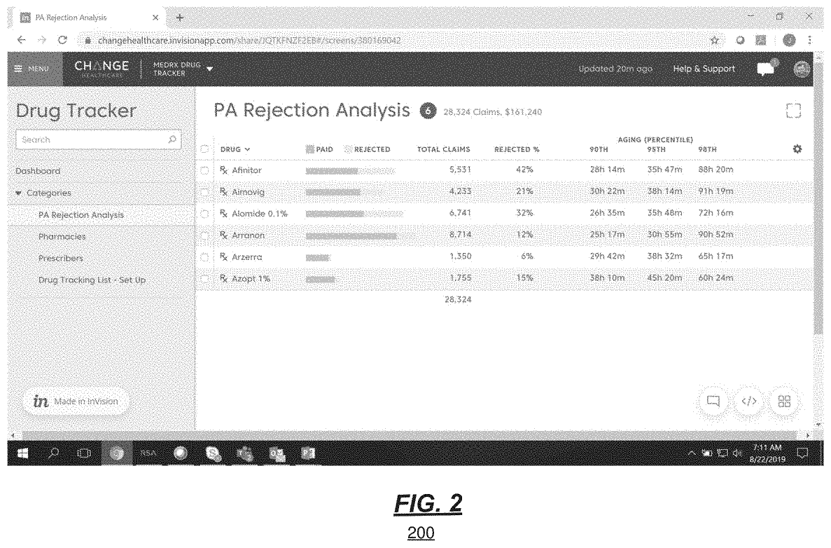

FIG. 2 is an illustration of a graphical user interface (GUI) 200 that may be used to implement the portal 123. As shown, the GUI 200 may provide a variety of metrics regarding prior authorizations (PAs) for various drugs associated with a drug manufacturer 103. In the example shown, the GUI 200 is being used to view metrics for each drug including total number of claims 107 received, the total number of claims 107 accepted (paid), and the total number of claims 107 that have been rejected due to missing or incomplete prior authorization requests 114. The GUI 200 may be further used to show metrics related to individual providers 101 (pharmacies), prescribers (physicians), and to select the particular drugs that are tracked for the manufacturer 103. As will be discussed further below, in some embodiments, the manufacturers 103 may be able to schedule training for a particular provider 101 through the GUI 200.

Returning to FIG. 1, the training engine 123 may monitor the various metrics associated with claims 107 and specifically the rate of claim 107 denial due to missing or incomplete prior authorization requests 114. The metrics that trigger training for each specialty drug and/or manufacturer 103, as well as the type and form of training, may be stored by the training engine 121 as part of the training data 116.

In some embodiments, when an approval rate for claims 107 requiring prior authorization requests 114 for a particular specialty drug falls below a threshold percentage for a provider 101, the training engine 123 may schedule training for the associated provider 101. The training may be training on how to best complete the paperwork associated with the prior authorization request 114 for the specialty drug so that the approval rate for claims 107 for the specialty drug increases. Alternatively, the training engine 123 may schedule training when the total number of claim 107 denials due to prior authorization requests 114 passes a threshold number. The threshold percentage or threshold number that triggers training may be provided by the manufacturer 103 associated with the specialty drug.

In some embodiments, the training engine 123 may schedule training in a variety of ways. As one example, the training engine 123 may send the provider 101 a link or invitation to view a training video associated with the specialty drug. The training video may have been created by the manufacturer 103. As another example, the training engine 123 may schedule an in-person visit to the provider 101 with instructors associated with the manufacturer 103. Other types of training may be supported.

In some embodiments, the training engine 123 may schedule training with a provider 101 automatically when one or more approval rates for a specialty drug falls below a threshold. Alternatively, the training engine 123 may alert the manufacturer 103 through the portal 123, and the manufacturer 103 may initiate the training process using the portal 123.

In some embodiments, the manufacturer 103 may provide payments 105 to the training engine 123 in exchange for scheduling training with providers 101 or for monitoring the metrics associated with their specialty drugs. For example, the manufacturer 103 may provide a flat monthly payment to the training engine 123 for monitoring each specialty drug and provider 101, may provide a payment 105 for scheduling training for a provider 103, or may provide payment based on measured improvements to the acceptance metrics achieved for each provider 103 by the training engine 123. The training engine 121 and the manufacturer 103 may periodically negotiate payment terms for the monitoring and training services provided by the training engine 123 to the manufacturer 103.

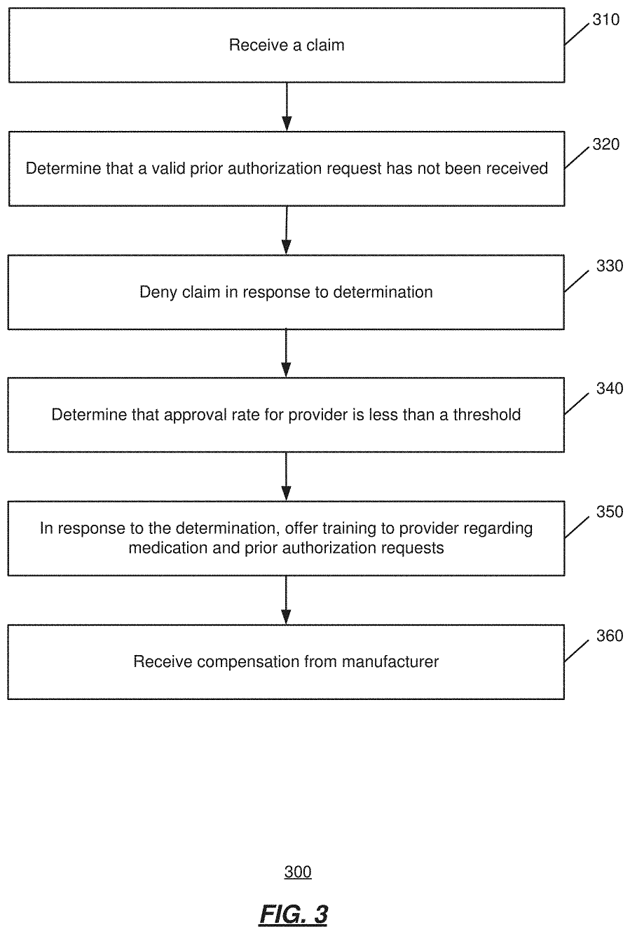

FIG. 3 is an illustration of a method 300 for providing training to providers to improve claim acceptance rates for specialty drugs. The method 300 may be implemented by the claim system 110.

At 310, a claim is received. The claim 107 may be received by the claim system 110 from a provider 101 such as a pharmacy. The claim 107 may be a claim for a specialty medication. Depending on the embodiment, the claim 107 may be a request for payment for the specialty medication from a third-party payor 102 such as an insurance company.

At 320, that a valid prior authorization request has not been received is determined. The claim engine 120 may determine that the valid prior authorization request 114 has not been received. As described above, each specialty medication may require that a prior authorization request 114 be received for a patient before a claim 107 can be approved. Depending on the embodiment, the prior authorization request 114 may include a list of documents or certain minimum facts or standards that must be met for the third-party payor 102 to approve the claim 107 for the medication. The prior authorization requests 114 are typically completed by the doctor that prescribed the specialty medication but may also be completed by the pharmacist or provider 101 that is generating the claim 107.

At 330, the claim is denied in response to the determination. The claim 107 may be denied by the claim engine 120 by sending a denial 111 to the provider 101. The denial 111 may include a code or indicator that shows that the claim 107 was denied due to an issue with the prior authorization request 114. Alternatively, the claim denial may be received from a claim processing system due to the lack of the valid prior authorization request.

At 340, that the approval rate for the provider is below a threshold is determined. The determination may be made by the monitoring engine 121 using a threshold provided by a manufacturer 103 associated with the specialty drug indicated by the claim 107. The approval rate for the provider 101 being below the threshold may indicate that the provider 101 may be having difficulty preparing or completing prior authorization requests 114 for the specialty drug indicated by the claim 107.

At 350, in response to the determination, training may be offered to the provider regarding the specialty medication and prior authorization 114 requests. The training may be offered by the training engine 123 according to information associated with the specialty drug and/or the manufacturer 103 stored in the training data 116. For example, the training engine 123 may provide an email or link that the provider 101 may use to receive electronic training on the specialty drug and the prior authorization process.

At 360, compensation is received from the manufacturer. The compensation or payment 105 may be provided by the manufacturer 103 in exchange for the training provided by the training engine 123 or in exchange for determining that the acceptance rate for the provider 101 is below the approval rate.

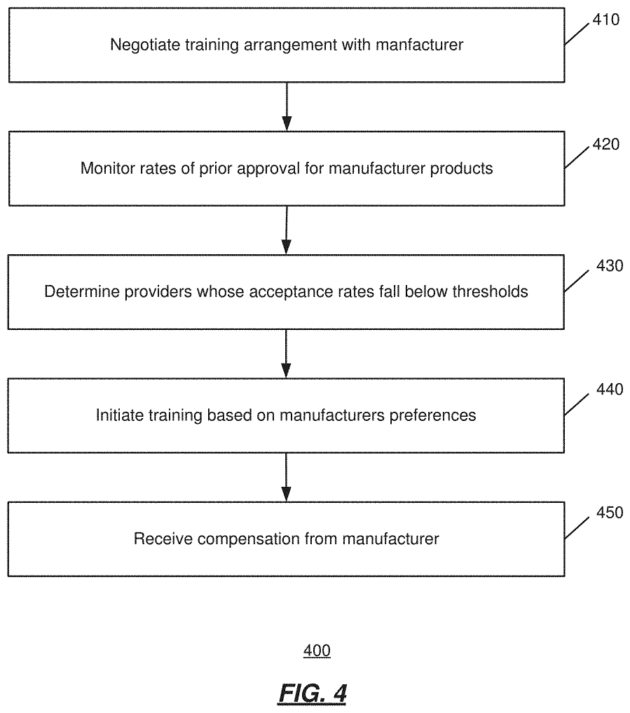

FIG. 4 is an illustration of a method 400 for compensating an entity for monitoring prior authorization approval rates. The method 400 may be implemented by the prior authorization system 110.

At 410, a training arrangement is negotiated with a manufacturer 103. The training arrangement may be negotiated by the claim system 110 with the manufacturer 103. The manufacturer 103 may be drug company and may manufacture several specialty drugs. The training arrangement may be for the monitoring engine 121 to monitor metrics associated with the specialty drugs and the approval rate of one or more more providers 101 who submit requests 107 for prior authorization. The training arrangement may specify the type of training that is provided (e.g., online or in-person), the threshold acceptance rate that will trigger training, and the payment 105 that the manufacturer 103 will provide for the training. Depending on the embodiment, rather than pay for the training, the training arrangement may specify a monthly payment 105 for the monitoring of the metrics and the manufacturer 103 may train the providers 101 themselves.

At 420, the approval or failure rates for claims 107 for a manufacturer 103 are monitored. The rates (e.g., metrics) may be monitored by the monitoring engine 121. In particular, the monitoring engine 121 may monitor the failure rates of claims 107 due to incomplete or missing prior authorization requests 114.

At 430, providers with acceptance rates that fall below thresholds are determined. The acceptance rates may be determined by the training engine 123 using the thresholds provided by the manufacturer 103. A low acceptance rate may indicate that the provider 101 needs more training or help preparing the associated prior authorization requests 114.

At 440, training may be initiated based on manufacturer preferences. Depending on the embodiment, the training may include sending a link or video that includes training related to the particular specialty drug. The training materials may have been generated by the manufacturer 103 or by the training engine 123. Initiating the training may further include arranging for in-person training at a location selected by the provider 101 and/or the manufacturer 103.

At 450, compensation is received from the manufacturer. The compensation may be received by the claim system 110 from the manufacturer 103. The payment 105 may have been specified by the training arrangement negotiated at 410.

FIG. 5 shows an exemplary computing environment in which example embodiments and aspects may be implemented. The computing device environment is only one example of a suitable computing environment and is not intended to suggest any limitation as to the scope of use or functionality.

Numerous other general purpose or special purpose computing devices environments or configurations may be used. Examples of well-known computing devices, environments, and/or configurations that may be suitable for use include, but are not limited to, personal computers, server computers, handheld or laptop devices, multiprocessor systems, microprocessor-based systems, network personal computers (PCs), minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 5, an exemplary system for implementing aspects described herein includes a computing device, such as computing device 500. In its most basic configuration, computing device 500 typically includes at least one processing unit 502 and memory 504. Depending on the exact configuration and type of computing device, memory 504 may be volatile (such as random access memory (RAM)), non-volatile (such as read-only memory (ROM), flash memory, etc.), or some combination of the two. This most basic configuration is illustrated in FIG. 5 by dashed line 506.

Computing device 500 may have additional features/functionality. For example, computing device 500 may include additional storage (removable and/or non-removable) including, but not limited to, magnetic or optical disks or tape. Such additional storage is illustrated in FIG. 5 by removable storage 508 and non-removable storage 510.

Computing device 500 typically includes a variety of computer readable media. Computer readable media can be any available media that can be accessed by the device 500 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 504, removable storage 508, and non-removable storage 510 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 500. Any such computer storage media may be part of computing device 500.

Computing device 500 may contain communication connection(s) 512 that allow the device to communicate with other devices. Computing device 500 may also have input device(s) 514 such as a keyboard, mouse, pen, voice input device, touch input device, etc. Output device(s) 516 such as a display, speakers, printer, etc. may also be included. All these devices are well known in the art and need not be discussed at length here.

It should be understood that the various techniques described herein may be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Although exemplary implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be effected across a plurality of devices. Such devices might include personal computers, network servers, and handheld devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A method comprising:
receiving a claim from a provider by a computing device, wherein the claim is for a drug produced by a manufacturer;
transmitting the claim to a claim processing system;
receiving a claim denial response from the claim processing system;
updating one or more metrics for the provider and the drug based on the claim denial response by the computing device;
determining whether the one or more metrics fall below a threshold for the manufacturer and the drug by the computing device; and
if it is determined that the one or more metrics fall below the threshold, initiating training for the provider by the computing device.

2. The method of claim 1, wherein the drug is a specialty drug.

3. The method of claim 1, further comprising receiving compensation for the training by the manufacturer.

4. The method of claim 1, wherein at least one metric of the one or more metrics is an approval rate.

5. The method of claim 1, wherein initiating training comprises providing a link to training materials to the provider.

6. The method of claim 1, wherein the provider is a pharmacy or a physician.

7. The method of claim 1, wherein the training is training on completing paperwork related to a prior authorization.

8. A method comprising:
monitoring acceptances and denials of claims due to prior authorization requests for a plurality of drugs associated with a manufacturer by a computing device, wherein each claim is associated with a provider of a plurality of providers;
for each provider of the plurality of providers and each drug of the plurality of drugs, computing an acceptance rate for the claims for the drug and the provider by the computing device;
determining that a computed acceptance rate for a first drug of the plurality drugs and a first provider of the plurality of providers falls below a threshold by the computing device; and
in response to the determination, initiating training for the first provider on preparing prior authorization requests for the first drug by the computing device.

9. The method of claim 8, wherein the drug is a specialty drug.

10. The method of claim 8, further comprising receiving compensation for the training by the manufacturer.

11. The method of claim 8, wherein initiating training comprises providing a link to training materials to the first provider.

12. The method of claim 11, further comprising receiving the training materials from the manufacturer.

13. The method of claim 8, wherein the first provider is a pharmacy or a physician.

14. The method of claim 8, wherein the training is training on completing paperwork related to the prior authorization request for the first drug.

15. The method of claim 8, further comprising:
providing a graphical user interface to the manufacturer; and
displaying some or all of the computed acceptance rates for the plurality of drugs for some or all of the plurality of providers in the graphical user interface.

16. The method of claim 8, further comprising receiving the threshold from the manufacturer.

17. A system comprising:
at least one computing device; and
a memory storing instructions that when executed by the at least one computing device cause the at least one computing device to:
receive a claim from a provider, wherein the claim is for a drug produced by a manufacturer;
transmit the claim to a claim processing system;
receive a claim denial response from the claim processing system;
update one or more metrics for the provider and the drug based on the claim denial response;
determine whether the one or more metrics fall below a threshold for the manufacturer and the drug; and
if it is determined that the one or more metrics fall below the threshold, initiate training for the provider.

18. The system of claim 17, wherein the drug is a specialty drug.

19. The system of claim 17, further comprising receiving compensation for the training by the manufacturer.

20. The system of claim 17, wherein the metric is an approval rate.
